(19) United States
(12) Patent Application Publication
(10) Pub. No.: US 2024/0021268 A1
(43) Pub. Date: Jan. 18, 2024

(54) GENE MODEL FOR JUDGING PROGNOSIS OF HEPATOCELLULAR CARCINOMA PATIENTS, CONSTRUCTION METHOD AND USE THEREOF

(71) Applicant: ZHEJIANG UNIVERSITY, Hangzhou, Zhejiang (CN)

(72) Inventors: Junjie XU, Hangzhou (CN); Xiujun CAI, Hangzhou (CN); Qijiang MAO, Hangzhou (CN); Haoqi PAN, Hangzhou (CN); Xiao LIANG, Hangzhou (CN)

(21) Appl. No.: 18/358,001

(22) Filed: Jul. 24, 2023

Related U.S. Application Data
(63) Continuation of application No. PCT/CN2021/139502, filed on Dec. 20, 2021.

(30) Foreign Application Priority Data
Sep. 16, 2021 (CN) 202111089547.X

(51) Int. Cl. G16B 20/10; G16B 40/20; G16H 50/30

(52) U.S. Cl. CPC G16B 20/10; G16B 40/20; G16H 50/30

(57) ABSTRACT

Disclosed are a gene model for judging prognosis of hepatocellular carcinoma and a construction method and use thereof. According to the present invention, genes with differential expression are obtained by comparing data of hepatocellular carcinoma patient samples with transcriptome data of normal patient samples, and after integration with an extracellular matrix gene set, a LASSO-COX regression model is reduced to obtain a model of 18 genes. The model of the present invention can evaluate the prognosis of hepatocellular carcinoma patients, distinguish and select patients with poor prognosis, so as to guide clinicians to provide more active treatment schemes, and meanwhile avoid over treatment of low-risk hepatocellular carcinoma patients. The gene model helps to construct a tissue chip based on extracellular matrix genes, which can quickly evaluate the prognosis of the hepatocellular carcinoma patients after surgery and realize clinical transformation.

### CROSS-REFERENCE TO RELATED APPLICATIONS

The present application is a National Stage of International Application No. PCT/CN2021/139502, filed on Dec. 20, 2021, which claims priority to Chinese Patent Application No. 202111089547.X, filed on Sep. 16, 2021, both of which are hereby incorporated by reference in their entireties.

### REFERENCE TO SEQUENCE LISTING

The present application is being filed along with a Sequence Listing in electronic format. The Sequence Listing is provided as a file entitled DF223160US-SEQUENCE LISTING ST.26, created on Mar. 23, 2023, which is approximately 22.5 Kb in size. The information in the electronic format of the Sequence Listing is incorporated herein by reference in its entirety.

### TECHNICAL FIELD

The present invention belongs to the technical field of biomedicine, and particularly relates to a gene model for judging prognosis of hepatocellular carcinoma patients and use thereof.

### BACKGROUND

Hepatoma is one of the ten most common malignant tumors in the world. There are about 500,000 new cases worldwide every year, of which hepatocellular carcinoma accounts for 85%. With the promotion of tumor markers and imaging examinations, the level of surgery and the development of various new treatment methods such as intra-arterial chemoembolization, the 5-year survival rate of hepatic cell carcinoma (HCC) has been improved. But overall, the prognosis of hepatocellular carcinoma remains unsatisfactory. One of the main reasons is the lack of effective markers for predicting the prognosis of the hepatocellular carcinoma patients, which makes it impossible to stratify the risk of the hepatocellular carcinoma patients and to guide clinicians to conduct early intervention and early treatment for high-risk hepatocellular carcinoma patients. Current research shows that the tumor microenvironment, especially the extracellular matrix, can promote tumor growth, invasion and metastasis, and has a great impact on the prognosis of tumor patients.

### SUMMARY

Aiming at the lack of effective markers for judging prognosis of hepatocellular carcinoma patients in the current clinic, and the prognosis of the hepatocellular carcinoma patients cannot be judged, according to the present invention, a gene combination model is constructed from a gene level to evaluate the prognosis of the hepatocellular carcinoma patients, the extracellular matrix-related genes of the hepatocellular carcinoma patients are integrated and analyzed to construct a related gene combination model, and a tissue chip based on extracellular matrix genes is constructed, which can realize that the prognosis of the hepatocellular carcinoma patients is evaluated through risk scores. The results obtained from the evaluation help clinicians to stratify the hepatoma patients and provide the possibility for precise treatment of the hepatocellular carcinoma patients.

The scheme that the present invention adopts is specifically as follows:

A construction method of a gene model for judging prognosis of hepatocellular carcinoma patients includes the following steps:
(1) obtaining transcriptome data of hepatocellular carcinoma and normal liver tissue samples, comparing differential genes in the data of the hepatocellular carcinoma tissue samples and the data of the normal liver tissue samples, setting P-value<0.05 to obtain genes with significant differences, and integrating the genes with significant differences with an extracellular matrix gene set (559 extracellular matrix-related genes); and
(2) using a LASSO method for analysis later, using 1000 Cox LASSO regression iterations and 10-fold cross-validation based on an R language glmnet package to reduce seed genes to 18 ECM gene sets related to HCC prognosis, including: 18 gene combinations (Table 1) of MMP1, EPO, MMRN1, S100A9, ADAM9, GPC1, SPP1, GLDN, FGF9, CXCL5, CST7, THBS3, ANXA10, PIK3IP1, MMP25, CLEC3B, PZP and CLEC17A, and using the 18 genes as markers to construct a risk score model for obtaining prognosis prediction of hepatocellular carcinoma.

A gene model obtained by construction of the above construction method, is specifically:
a risk score of hepatocellular carcinoma patients=(0.069*MMP1 expression level)+(0.049*EPO expression level)+(0.042*MMRN1 expression level)+(0.036*S100A9 expression level)+(0.027*ADAM9 expression level)+(0.024*GPC1 expression level)+(0.021*SPP1 expression level)+(0.014*GLDN expression level)+(0.007*FGF9 expression level)+(0.001*CXCL5 expression level)−(0.024*CST7 expression level)−(0.027*THBS3 expression level)−(0.042*ANXA10 expression level)−(0.049*PIK3IP1 expression level)−(0.051*MMP25 expression level)−(0.054*CLEC3B expression level)−(0.062*PZP expression level)−(0.069*CLEC17A expression level).

Further, a TCGA database is used as a training set, a GEO database and an ICGC database are used as verification sets, the risk score of the gene model is analyzed, and the gene model is verified through CLIP staging and TMN staging, which shows that the risk scores of the hepatocellular carcinoma patients are related to a survival period, and a patient with a high-risk score has a short survival period and poor prognosis.

Use of a gene model in evaluating prognosis of hepatocellular carcinoma.

A tissue chip based on extracellular matrix genes, including probes for detecting MMP1, EPO, MMRN1, S100A9, ADAM9, GPC1, SPP1, GLDN, FGF9, CXCL5, CST7, THBS3, ANXA10, PIK3IP1, MMP25, CLEC3B, PZP and CLEC17A. It provides a possibility to provide precise treatment for hepatocellular carcinoma patients, which can quickly evaluate the prognosis of the hepatocellular carcinoma patients after surgery and realize clinical transformation.

The present invention has the beneficial effects that the gene combination model with 18 genes is constructed, the tissue chip based on the extracellular matrix genes can be constructed through the gene model, the prognosis of the hepatocellular carcinoma patients can be evaluated, hepatocellular carcinoma patients with poor prognosis can be distinguished and selected, that is, the hepatocellular carcinoma patients are stratified, the hepatocellular carcinoma patients with high risks and poor prognosis are selected, so as to guide clinicians to provide more active treatment schemes for the high-risk patients, and meanwhile avoid over treatment of low-risk hepatocellular carcinoma patients.

### BRIEF DESCRIPTION OF DRAWINGS

The present invention is further illustrated below in conjunction with the accompanying drawings and embodiments.

FIG. 1 is a differential gene volcano diagram of a gene model of the present invention.

FIG. 2 is a LASSO-Cox regression model construction diagram of a gene model of the present invention.

FIG. 3 is a combined gene model diagram of 18 genes of the present invention.

FIG. 4 is a risk score distribution diagram of hepatocellular carcinoma patients in a training set TCGA, in which an abscissa is a serial number of patients increasing according to risk scores, and a dotted line is a cut-off value.

FIG. 5 is a survival period distribution diagram of hepatocellular carcinoma patients in a training set TCGA, in which an abscissa is a serial number of patients increasing according to risk scores, a dotted line near 190 is a cut-off value, and a line near 120 and 250 is a dividing line between death and survival.

FIG. 6 is a survival period distribution diagram of hepatocellular carcinoma patients in a verification set GEO, in which an abscissa is a serial number of patients increasing according to risk scores, and a dotted line is a cut-off value.

FIG. 7 is a survival period distribution diagram of hepatocellular carcinoma patients in a verification set ICGC, in which an abscissa is a serial number of patients increasing according to risk scores, and a dotted line is a cut-off value.

FIG. 8 is a risk score result diagram of different CLIP stages of hepatocellular carcinoma patients in a training set TCGA.

FIG. 9 is a risk score result diagram of different TMN stages of hepatocellular carcinoma patients in a training set TCGA.

FIG. 10 is a relational diagram of prognosis and survival period of hepatocellular carcinoma patients after grouping based on a gene model of the present invention in a training set TCGA.

FIG. 11 is a result diagram of sensitivity and specificity of prognosis of hepatocellular carcinoma patients grouped based on a gene model of the present invention in a training set TCGA.

FIG. 12 a relational diagram of prognosis and survival period of hepatocellular carcinoma patients grouped based on a gene model of the present invention in a verification set GEO.

FIG. 13 is a relational diagram of prognosis and survival period of hepatocellular carcinoma patients grouped based on a gene model of the present invention in a verification set ICGA.

### DESCRIPTION OF EMBODIMENTS

The present invention provides a gene model for predicting prognosis of hepatocellular carcinoma based on extracellular matrix genes and use thereof. That is, aiming at the differential genes of the extracellular matrix of the hepatocellular carcinoma patients, a risk model of prognosis of hepatocellular carcinoma is established by using data of hepatocellular carcinoma tissue samples and normal liver tissue samples in the database and statistical analysis, which can be used as a gene model for predicting the prognosis of hepatocellular carcinoma, so that a tissue chip based on extracellular matrix genes is constructed, which is helpful for evaluating the prognosis of hepatocellular carcinoma patients after surgery. The inclusion and exclusion criteria for hepatocellular carcinoma tissue samples are:
(1) have not received other cancer malignant tumors;
(2) no history of other malignant tumors; and
(3) have complete clinical pathological data and follow-up information.

The effects of the present invention will be further described below in conjunction with specific embodiments.

### Embodiment 1: Construction of a Gene Model for Judging Prognosis of Hepatocellular Carcinoma Patients

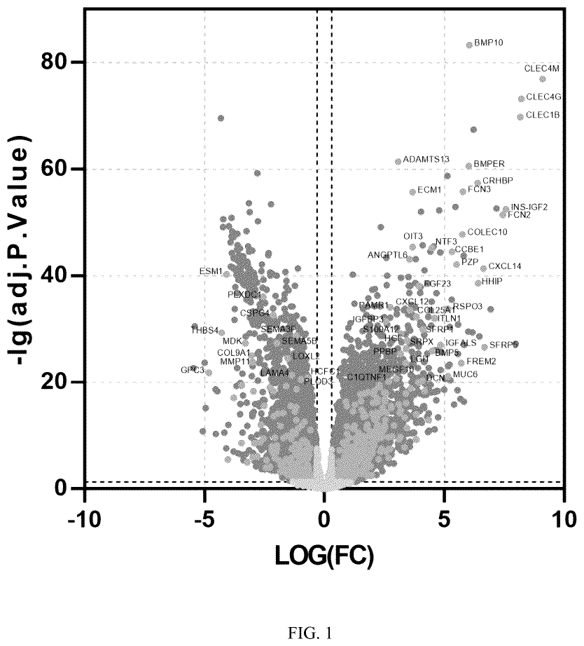

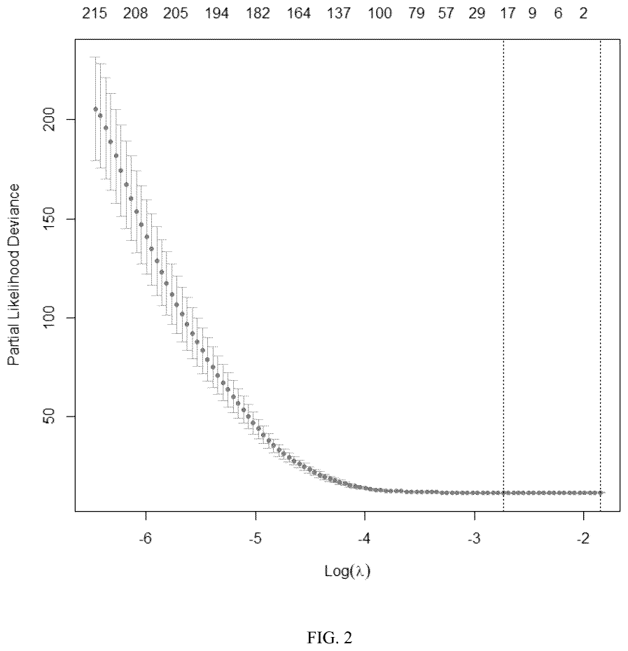

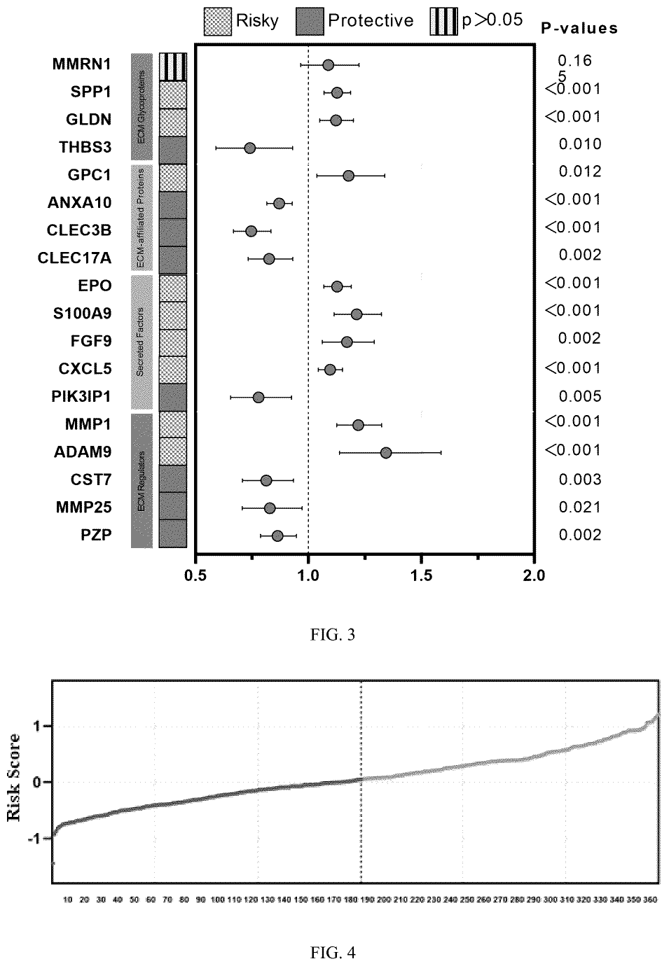

The gene model of the present invention for judging the prognosis of the hepatocellular carcinoma patients is constructed and obtained by the following steps:
(1), transcriptome data of 371 hepatocellular carcinoma tissue samples and 50 normal liver tissue samples and clinical information of corresponding patients (including gender, overall survival time, survival status, etc.) are downloaded from a TCGA database (https://portal.gdc.cancer.gov/), differential genes in the data of the hepatocellular carcinoma tissue samples and the data of the normal liver tissue samples in the TCGA database are compared, P-value<0.05 is set to obtain genes with significant differences, and the genes with significant differences are integrated with 559 extracellular matrix (ECM)-related genes (see FIG. 1).
(2) a LASSO method is used for analysis later, 1000 Cox LASSO regression iterations and 10-fold cross-validation are used based on an R language glmnet package to select 18 candidate genes related to the ECM with statistic significance and prognosis AUC and HR values of these genes (see Table 1 and FIG. 2). Coefficients of a Cox LASSO regression model are used as weights, and a risk score model (see FIG. 3) of prognosis prediction of hepatocellular carcinoma based on 18 genes including MMP1, EPO, MMRN1, S100A9, ADAM9, GPC1, SPP1, GLDN, FGF9, CXCL5, CST7, THBS3, ANXA10, PIK3IP1, MMP25, CLEC3B, PZP and CLEC17A as markers is constructed.

The risk score model of prognosis prediction of hepatocellular carcinoma is specifically: a risk score of hepatocellular carcinoma patients=(0.069*MMP1 expression level)+(0.049*EPO expression level)+(0.042*MMRN1 expression level)+(0.036*S100A9 expression level)+(0.027*ADAM9 expression level)+(0.024*GPC1 expression level)+(0.021*SPP1 expression level)+(0.014*GLDN expression level)+(0.007*FGF9 expression level)+(0.001*CXCL5 expression level)−(0.024*CST7 expression level)−(0.027*THBS3 expression level)−(0.042*ANXA10 expression level)−(0.049*PIK3IP1 expression level)−(0.051*MMP25 expression level)−(0.054*CLEC3B expression level)−(0.062*PZP expression level)−(0.069*CLEC17A expression level).

TABLE 1
18 ECM genes obtained after the LASSO regression model

| Gene name | Disease | AUC | HR |
|---|---|---|---|
| MMP1 | Hepatoma | 0.628 | 1.220 |
| EPO | Hepatoma | 0.607 | 1.127 |
| MMRN1 | Hepatoma | 0.542 | 1.088 |
| S100A9 | Hepatoma | 0.589 | 1.213 |
| ADAM9 | Hepatoma | 0.586 | 1.344 |
| GPC1 | Hepatoma | 0.639 | 1.178 |
| SPP1 | Hepatoma | 0.614 | 1.127 |
| GLDN | Hepatoma | 0.603 | 1.122 |
| FGF9 | Hepatoma | 0.555 | 1.171 |
| CXCL5 | Hepatoma | 0.575 | 1.096 |
| CST7 | Hepatoma | 0.564 | 0.813 |
| THBS3 | Hepatoma | 0.623 | 0.741 |
| ANXA10 | Hepatoma | 0.629 | 0.870 |
| PIK3IP1 | Hepatoma | 0.559 | 0.779 |
| MMP25 | Hepatoma | 0.549 | 0.829 |
| CLEC3B | Hepatoma | 0.610 | 0.746 |
| PZP | Hepatoma | 0.608 | 0.863 |
| CLEC17A | Hepatoma | 0.593 | 0.826 |

### Embodiment 2: Use of a Risk Score Model of Prognosis Prediction of Hepatocellular Carcinoma in Evaluating Prognosis of Hepatocellular Carcinoma

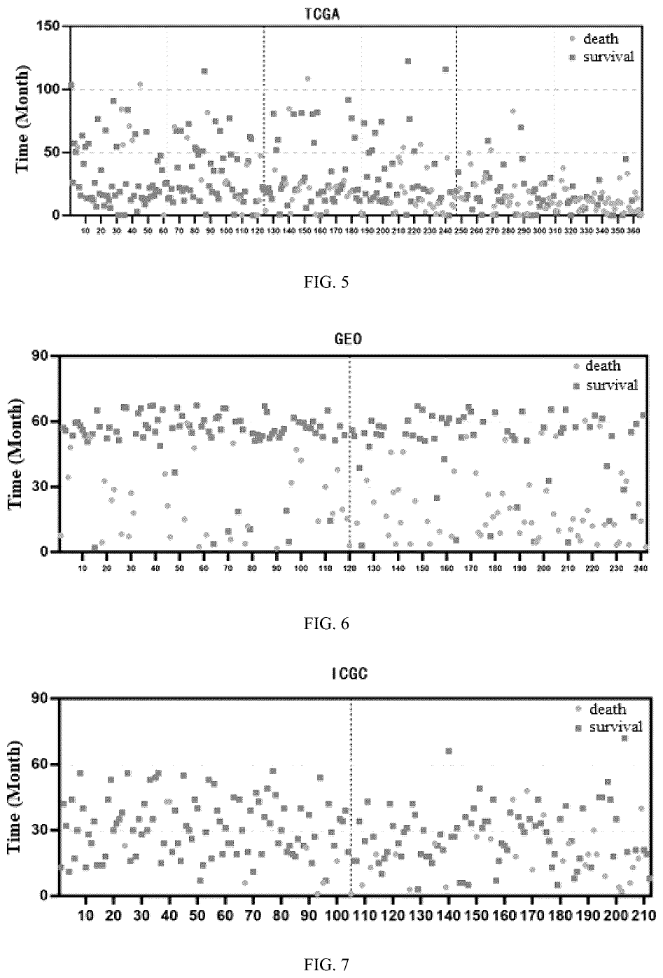

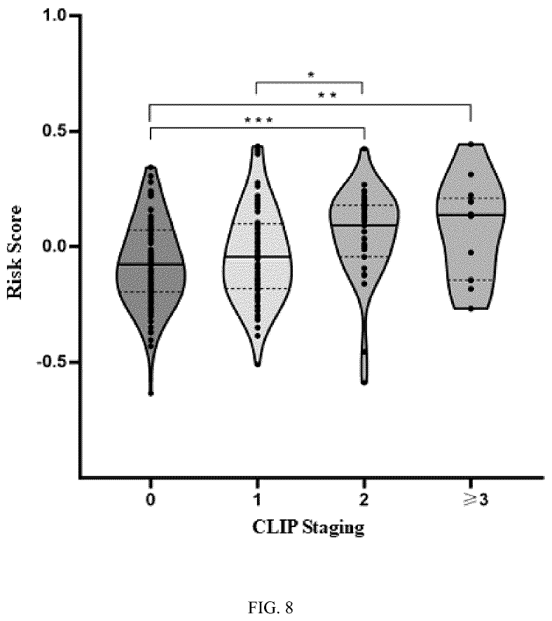

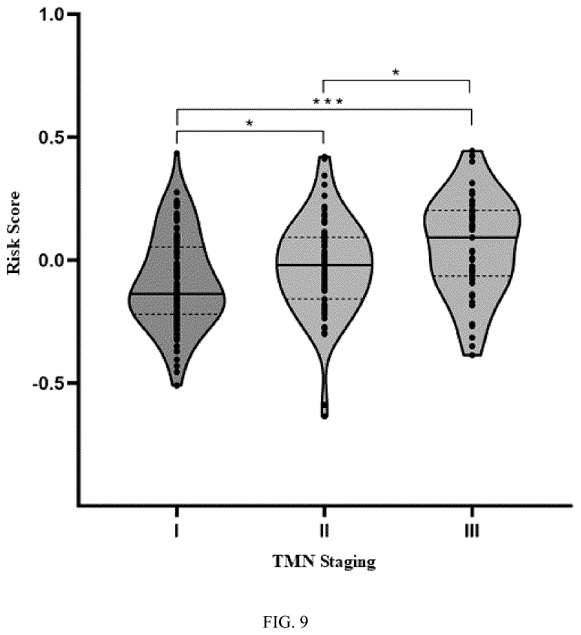

Transcriptome data of 371 hepatocellular carcinoma tissue samples in the TCGA database are used as a training set, data of 247 hepatocellular carcinoma tissues in GSE140520 of a GEO database (https://www.ncbi.nlm.nih.gov/geo/) and 203 hepatocellular carcinoma tissues of an ICGC database (https://daco.icgc.org/) are used as verification sets, scores of each hepatocellular carcinoma patient in the training set are calculated respectively according to the risk score model, a median (0.044954) of the scores is taken as a cut-off value, the scores are divided into a high-risk value group and a low-risk value group, the relational diagrams (FIGS. 4-8) of risk score and survival period, as well as CLIP staging and TMN staging of patients in the two groups are drawn, and the effects of the risk score model of prognosis prediction of hepatocellular carcinoma are verified. FIG. 4 and FIG. 5 are risk score distribution diagram and survival period distribution diagram of hepatocellular carcinoma patients in the training set TCGA according to the cut-off value, FIG. 6 and FIG. 7 are survival period diagrams of hepatocellular carcinoma patients in the verification sets GEO and ICGC according to the cut-off value, FIG. 8 and FIG. 9 are risk score results of different CLIP stage and TMN stage of hepatocellular carcinoma patients in the training set TCGA, it can be seen that the higher the risk scores, the higher the survival rate of the patients, and the higher the CLIP stage and TMN stage, which shows that the model has a good hepatocellular carcinoma typing effect.

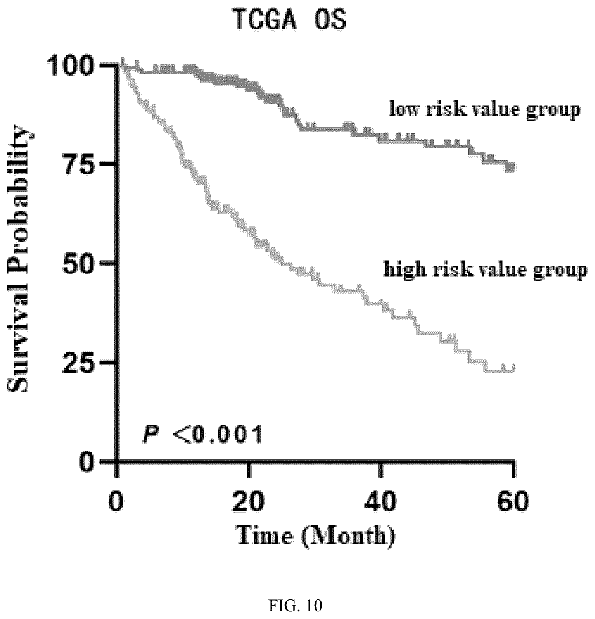

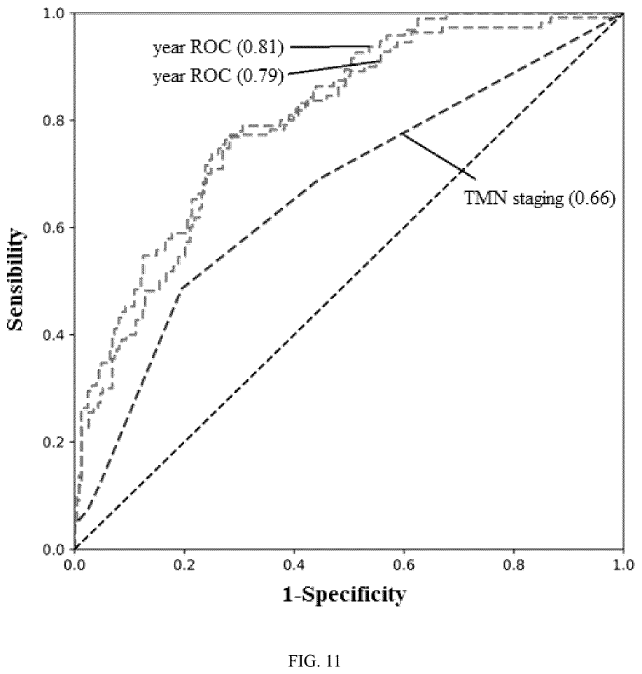

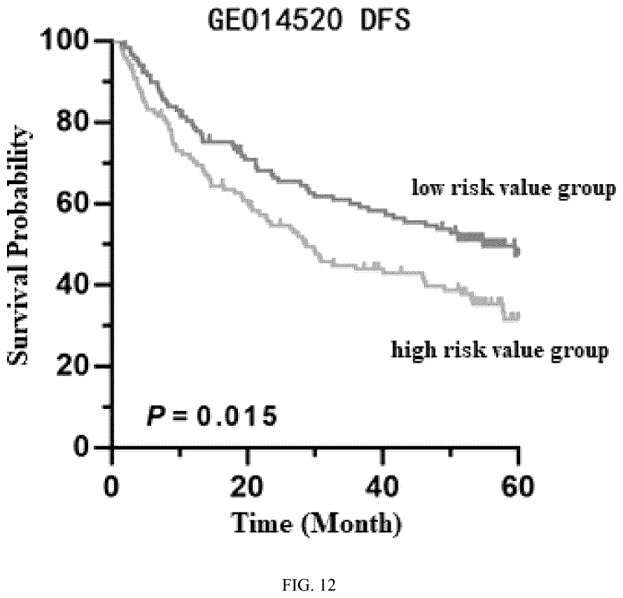

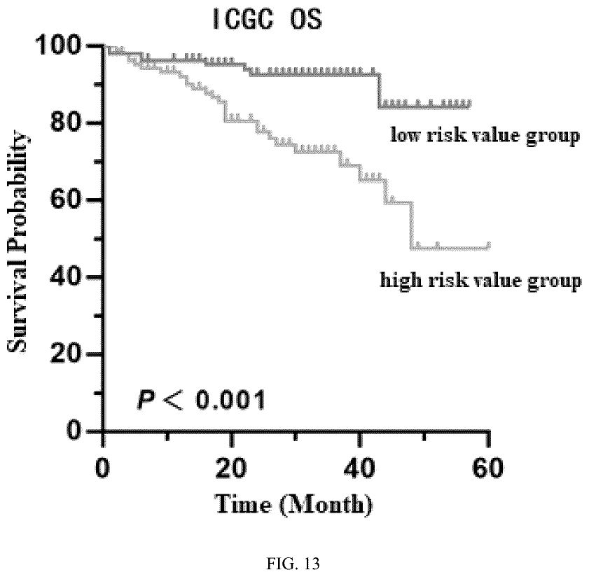

Further, a prediction performance of the model is evaluated through an ROC curve, FIG. 10 is a relational diagram of prognosis and survival period of hepatocellular carcinoma patients in the database of the training set TCGA, in which the survival period of the hepatocellular carcinoma patients in the high-risk value group is short, and prognosis is worse than that in the low-risk value group (see FIG. 10). FIG. 11 and Table 2 are results of sensitivity and specificity of prognosis of HCC verified by the model, 3-year AUC of the risk model is 0.81, sensitivity is 73.7%, and specificity is 75%; and 5-year AUC is 0.79, sensitivity is 77.3%, and specificity is 71.7%. Data of 247 hepatocellular carcinoma tissues in GSE140520 of the GEO database (https://www.ncbi.nlm.nih.gov/geo/) and 203 hepatocellular carcinoma tissues in the ICGC database are used as the verification sets for verification (see FIG. 12 and FIG. 13), the results are consistent with the results in the TCGA database. Table 3 is the results of sensitivity and specificity of prognosis of HCC verified by the model in the GEO database, 3-year AUC of the risk model is 0.626, sensitivity is 68.8%, and specificity is 55.8%; and 5-year AUC is 0.625, sensitivity is 60.0%, and specificity is 34.7%. Table 4 is the results of sensitivity and specificity of prognosis of HCC verified by the model in the ICGC database, 3-year AUC of the risk model is 0.723, sensitivity is 93.3%, and specificity is 52.7%; 5-year AUC is 0.717, sensitivity is 88.9%, and specificity is 52.3%. The patients with high-risk scores have short survival periods and poor prognosis. It is shown that the risk score model of prognosis prediction of hepatocellular carcinoma of the present invention can be used for evaluating the prognosis of hepatocellular carcinoma.

TABLE 2
Test results of sensitivity and specificity
of a risk model in the TCGA database

| Risk model | AUC | Sensitivity | Specificity |
|---|---|---|---|
| 3-year ROC | 0.81 | 73.7% | 75.0% |
| 5-year ROC | 0.79 | 77.3% | 71.7% |

TABLE 3
Test results of sensitivity and specificity
of a risk model in the GEO database

| Risk model | AUC | Sensitivity | Specificity |
|---|---|---|---|
| 3-year ROC | 0.626 | 68.8% | 55.8% |
| 5-year ROC | 0.625 | 60.0% | 34.7% |

TABLE 4
Test results of sensitivity and specificity
of a risk model in the ICGC database

| Risk model | AUC | Sensitivity | Specificity |
|---|---|---|---|
| 3-year ROC | 0.723 | 93.3% | 52.7% |
| 5-year ROC | 0.717 | 88.9% | 52.3% |

The present invention further provides a gene chip, that is, probes for detecting 18 genes of MMP1, EPO, MMRN1, S100A9, ADAM9, GPC1, SPP1, GLDN, FGF9, CXCL5, CST7, THBS3, ANXA10, PIK3IP1, MMP25, CLEC3B, PZP and CLEC17A are constructed into the gene chip according to the above model, so as to facilitate clinical application. A sequence of each gene probe is preferably as shown in Table 5, and aiming at a plurality of probes of one gene, an average value of probe test results can be selected as a final expression level of the gene.

TABLE 5
Sequences of each gene probe of the gene chip

| Gene | Gene | |
|---|---|---|
| name | version | Gene sequence |
| MMP1 | HG19 | GGATCTGGAGATGGGCCGATAAAGTCAGT |
| | | ACGCAAAAGAAGAGTACGAAAGGACTAAA |
| | | CT (SEQ ID NO. 1) |
| EPO | HG19 | GAACCATGAAGACAGGATGGGGGCTGGCC |
| | | TCTGGCTCTCATGGGGTCCAAGTTTTGTG |
| | | TA (SEQ ID NO. 2) |
| MMRN1 | HG19 | GCTTGCATTTGAGTCTGAAAATATTAACA |
| | | GTGAAATACACTGTGATAGGGTTTTAACT |
| | | GG (SEQ ID NO. 3) |
| S100A9 | HG19 | CACAAATGCAGACAAGCAGCTGAGCTTCG |
| | | AGGAGTTCATCATGCTGATGGCGAGGCTA |
| | | AC (SEQ ID NO. 4) |
| ADAM9 | HG19 | ATTTCCGTTTCCATCATTGAATAAGTCTT |
| | | ATTCAGTCATCGGTGAGGTTAATGCACTA |
| | | AT (SEQ ID NO. 5) |
| GPC1 | HG19 | AGGTCCCCGGTTGCTGGTCAGGTCCCCAT |
| | | GGCTTGTTCTCTGGAACCTGACTTTAGAT |
| | | GT (SEQ ID NO. 6) |
| SPP1 | HG19 | TTTTGAAGATAAACCGAAACCTTCCAAAC |
| | | AGTCACTTCAGTCTTACCAAGAGGCTTTG |
| | | CA (SEQ ID NO. 7) |
| GLDN | HG19 | TCTTGGCCCCTGTGTGAATTCCTGCCTTT |
| | | CCCAGAAATGAGTCCAGGGTGTCTGACCT |
| | | CA (SEQ ID NO. 8) |
| | | GCACGTTGTTTACAACAACTCTCTCTACT |
| | | ACCACAAAGGGGGTTCTAATACCCTAGTG |
| | | AG (SEQ ID NO. 9) |
| FGF9 | HG19 | CAAAAGGACTGCGGCCTGATGCATGCTGG |
| | | AAAAAGACACGCTTTTCATTTCTGATCAG |
| | | TT (SEQ ID NO. 10) |
| CXCL5 | HG19 | TTTGCTGTTATTTTATCTGCTATGCTATT |
| | | GAAGTTTTGGCAATTGACTATAGTGTGAG |
| | | CC (SEQ ID NO. 11) |
| CST7 | HG19 | CATCACAAGGGCCCTAGTTCAGATAGTGA |
| | | AAGGCCTGAAATATATGCTGGAGGTGGAA |
| | | AT (SEQ ID NO. 12) |
| THBS3 | HG19 | GGGGCGTCTTGGTGTATTCTGCTTCTCCC |
| | | AAGAAAACATAATTTGGTCCAATCTCCAG |
| | | TA (SEQ ID NO. 13) |
| ANXA10 | HG19 | TGGTGATGCTGAGGACTACTAAAATGAAG |
| | | AGGACTTGGAGTACTGTGCACTCCTCTTT |
| | | CT (SEQ ID NO. 14) |
| PIK3IP1 | HG19 | TTTGTGTTCTGGTTAAAACCCTACCACTC |
| | | CCCCGCTTTTTTGGCGAATCCTAGTAAGA |
| | | GT (SEQ ID NO. 15) |
| | | AAGGAAAGATTGTAATCTCACCTCCAGAT |
| | | CCATAGGCTTCTGCTAGGAGCATGTTGCT |
| | | GA (SEQ ID NO. 16) |
| | | AAATCTCACCATATCCATTTGACTTAGGC |
| | | CTTTTGGAGTTAGGCAGAAGGGCCCTTCT |
| | | TC (SEQ ID NO. 17) |
| | | CCAACCTCAGTGGTTCAGTGGTGTCTGCA |
| | | AAATGCATCCATCCTGCCATCTGAGTAGC |
| | | AG (SEQ ID NO. 18) |
| | | ATGAGAAGACATGGAATGTTGGAAGCAGC |
| | | AACCGAAACAAAGCTGAAAACCTGTTGCG |
| | | AG (SEQ ID NO. 19) |
| | | TTTGTGTTCTGGTTAAAACCCTACCACTC |
| | | CCCCGCTTTTTTGGCGAATCCTAGTAAGA |
| | | GT (SEQ ID NO. 20) |
| MMP25 | HG19 | CTGTGTGTTCTCTGGATCTTTTCAGCCCT |
| | | GTGGTCCAGTGTCCATCACAGCCATGCTG |
| | | AC (SEQ ID NO. 21) |
| CLEC3B | HG19 | TCCTCTCCGTGCGCTTGGAGCCTCTTTTT |
| | | GCAAATAAAGTTGGTGCAGCTTCGCGGAG |
| | | AG (SEQ ID NO. 22) |
| PZP | HG19 | CTGGTTTTATTCCCCTGAAACCAACAGTA |
| | | AAAATGCTTGAAAGATCTAGCTCTGTGAG |
| | | CC (SEQ ID NO. 23) |
| CLEC17A | HG19 | ACATTGCCCGTGTAAGAGCTGACACCAAC |
| | | CAGTCCCTGGTGGAACTTTGGGGCTTATT |
| | | AG (SEQ ID NO. 24) |

The above description of the specific implementations is for the convenience of those ordinary skilled in the art to understand and use the present invention. It is obvious that those skilled in the art can easily make various modifications to these specific implementations and apply the general principles described here to other embodiments without creative labor. Therefore, the present invention is not limited to the above specific implementations. Improvements and modifications made by those skilled in the art according to the principles of the present invention without departing from the scope of the present invention should be within the protection scope of the present invention.

What is claimed is:

1. Use of a gene combination in preparing a tissue chip for judging prognosis of hepatocellular carcinoma, wherein
the tissue chip comprises a probe for detecting MMP1, EPO, MMRN1, S100A9, ADAM9, GPC1, SPP1, GLDN, FGF9, CXCL5, CST7, THBS3, ANXA10, PIK3IP1, MMP25, CLEC3B, PZP and CLEC17A, and a gene model for judging the prognosis of hepatocellular carcinoma is: a risk score of a hepatocellular carcinoma patient=(0.069*MMP1 expression level)+(0.049*EPO expression level)+(0.042*MMRN1 expression level)+(0.036*S100A9 expression level)+(0.027*ADAM9 expression level)+(0.024*GPC1 expression level)+(0.021*SPP1 expression level)+(0.014*GLDN expression level)+(0.007*FGF9 expression level)+(0.001*CXCL5 expression level)−(0.024*CST7 expression level)−(0.027*THBS3 expression level)−(0.042*ANXA10 expression level)−(0.049*PIK3IP1 expression level)−(0.051*MMP25 expression level)−(0.054*CLEC3B expression level)−(0.062*PZP expression level)−(0.069*CLEC17A expression level).

2. The use according to claim 1, wherein the risk score of the hepatocellular carcinoma patient is related to a survival period, and a patient with a high-risk score has a short survival period and poor prognosis.


SEQUENCE LISTING (24 sequences)

SEQ ID NO: 1 - DNA, 60 bp, synthetic construct
  source 1..60: mol_type = other DNA
ggatctggagatgggccgataaagtcagtacgcaaaagaagagtacgaaaggactaaact

SEQ ID NO: 2 - DNA, 60 bp, synthetic construct
  source 1..60: mol_type = other DNA
gaaccatgaagacaggatgggggctggcctctggctctcatggggtccaagttttgtgta

SEQ ID NO: 3 - DNA, 60 bp, synthetic construct
  source 1..60: mol_type = other DNA
gcttgcatttgagtctgaaaatattaacagtgaaatacactgtgatagggttttaactgg

SEQ ID NO: 4 - DNA, 60 bp, synthetic construct
  source 1..60: mol_type = other DNA
cacaaatgcagacaagcagctgagcttcgaggagttcatcatgctgatggcgaggctaac

SEQ ID NO: 5 - DNA, 60 bp, synthetic construct
  source 1..60: mol_type = other DNA
atttccgtttccatcattgaataagtcttattcagtcatcggtgaggttaatgcactaat

SEQ ID NO: 6 - DNA, 60 bp, synthetic construct
  source 1..60: mol_type = other DNA
aggtccccggttgctggtcaggtccccatggcttgttctctggaacctgactttagatgt

SEQ ID NO: 7 - DNA, 60 bp, synthetic construct
  source 1..60: mol_type = other DNA
ttttgaagataaaccgaaaccttccaaacagtcacttcagtcttaccaagaggctttgca

SEQ ID NO: 8 - DNA, 60 bp, synthetic construct
  source 1..60: mol_type = other DNA
tcttggcccctgtgtgaattcctgcctttcccagaaatgagtccagggtgtctgacctca

SEQ ID NO: 9 - DNA, 60 bp, synthetic construct
  source 1..60: mol_type = other DNA
gcacgttgtttacaacaactctctctactaccacaaagggggttctaataccctagtgag

SEQ ID NO: 10 - DNA, 60 bp, synthetic construct
  source 1..60: mol_type = other DNA
caaaaggactgcggcctgatgcatgctggaaaaagacacgcttttcatttctgatcagtt

SEQ ID NO: 11 - DNA, 60 bp, synthetic construct
  source 1..60: mol_type = other DNA
tttgctgttattttatctgctatgctattgaagttttggcaattgactatagtgtgagcc

SEQ ID NO: 12 - DNA, 60 bp, synthetic construct
  source 1..60: mol_type = other DNA
catcacaagggccctagttcagatagtgaaaggcctgaaatatatgctggaggtggaaat

SEQ ID NO: 13 - DNA, 60 bp, synthetic construct
  source 1..60: mol_type = other DNA
ggggcgtcttggtgtattctgcttctcccaagaaaacataatttggtccaatctccagta

SEQ ID NO: 14 - DNA, 60 bp, synthetic construct
  source 1..60: mol_type = other DNA
tggtgatgctgaggactactaaaatgaagaggacttggagtactgtgcactcctctttct

SEQ ID NO: 15 - DNA, 60 bp, synthetic construct
  source 1..60: mol_type = other DNA
tttgtgttctggttaaaaccctaccactcccccgcttttttggcgaatcctagtaagagt

SEQ ID NO: 16 - DNA, 60 bp, synthetic construct
  source 1..60: mol_type = other DNA
aaggaaagattgtaatctcacctccagatccataggcttctgctaggagcatgttgctga

SEQ ID NO: 17 - DNA, 60 bp, synthetic construct
  source 1..60: mol_type = other DNA
aaatctcaccatatccatttgacttaggccttttggagttaggcagaagggcccttcttc

SEQ ID NO: 18 - DNA, 60 bp, synthetic construct
  source 1..60: mol_type = other DNA
ccaacctcagtggttcagtggtgtctgcaaaatgcatccatcctgccatctgagtagcag

SEQ ID NO: 19 - DNA, 60 bp, synthetic construct
  source 1..60: mol_type = other DNA
atgagaagacatggaatgttggaagcagcaaccgaaacaaagctgaaaacctgttgcgag

SEQ ID NO: 20 - DNA, 60 bp, synthetic construct; identical to SEQ ID NO: 15
  source 1..60: mol_type = other DNA

SEQ ID NO: 21 - DNA, 60 bp, synthetic construct
  source 1..60: mol_type = other DNA
ctgtgtgttctctggatcttttcagccctgtggtccagtgtccatcacagccatgctgac

SEQ ID NO: 22 - DNA, 60 bp, synthetic construct
  source 1..60: mol_type = other DNA
tcctctccgtgcgcttggagcctctttttgcaaataaagttggtgcagcttcgcggagag

SEQ ID NO: 23 - DNA, 60 bp, synthetic construct
  source 1..60: mol_type = other DNA
ctggttttattcccctgaaaccaacagtaaaaatgcttgaaagatctagctctgtgagcc

SEQ ID NO: 24 - DNA, 60 bp, synthetic construct
  source 1..60: mol_type = other DNA
acattgcccgtgtaagagctgacaccaaccagtccctggtggaactttggggcttattag